(19) United States
(12) Patent Application Publication
(10) Pub. No.: US 2011/0123423 A1
(43) Pub. Date: May 26, 2011

(54) PHOTOCATALYTIC FLUIDIZED BED REACTOR WITH HIGH ILLUMINATION EFFICIENCY FOR PHOTOCATALYTIC OXIDATION PROCESSES

(76) Inventors: Paolo Ciambelli, Fisciano (SA) (IT); Diana Sannino, Fisciano (SA) (IT); Vincenzo Palma, Fisciano (SA) (IT); Vincenzo Vaiano, Boscoreale (NA) (IT)

(21) Appl. No.: 12/995,139

(22) PCT Filed: May 29, 2009
(86) PCT No.: PCT/IT09/00239

§ 371 Date: Feb. 9, 2011

(30) Foreign Application Priority Data
May 29, 2008 (IT) SA2008A000012

(51) Int. Cl. B01D 53/72; C07C 45/29; C07C 5/333

(52) U.S. Cl. 423/245.1; 422/146; 568/471; 585/442

(57) ABSTRACT

The invention relates to the realization of synthesis of organic compounds or abatement of volatile organic compounds (VOCs) in gas-solid fluidised bed photocatalytic reactor with improved illumination efficiency. The photoreactor consists of a two-dimensional fluidized bed catalytic reactor with two walls transparent to ultraviolet radiation, by an illumination system bases on a matrix of LEDs positioned near its external walls, and heated for Joule effect inside the catalytic bed to monitor the reaction temperature. Surprisingly, through the choice of a suitable catalyst and fluidized bed photoreactor operating conditions both total and partial oxidation reactions can be achieved with high activity and selectivity. Even more surprisingly, the value of the illuminated catalyst surface area per unit irradiated volume reaches values in the order of 10⁶ m⁻¹, significantly higher than those of microreactors, amounting to 250,000 m⁻¹ and slurry reactors with values in 8500-170000 m⁻¹.

The photocatalytic system reported in the present invention is shown to have high illumination efficiency due to the use of UV-LEDs, which, ensuring a direction of light irradiation direction orthogonal to the emission point, minimize the dispersion of photons.

### TECHNICAL FIELD OF THE INVENTION

The present invention concerns a gas-solid photocatalytic reactor with high illumination efficiency and its application to the removal of volatile organic compounds (VOCs) from gaseous streams, or to or innovative processes of synthesis of organic substances. The photoreactor has a low volume, with high illumination efficiency, and may be heated from the interior, up to 160° C. These features make it extremely versatile in installation and use.

### BACKGROUND OF THE INVENTION

When a photocatalytic reaction takes place, it is necessary to achieve an optimal exposure of the catalysts to light and a good contact between reactants and catalyst. To that aim, several reactor designs have been proposed (Van Gerven T., Mul G., Moulijn J., Stankiewicz A., Chem. Eng. Process, 46 (2007) 781). Slurry reactors, annular reactors, immersion reactors, optical tube reactors, optical fibres reactors and microreactors are among the most cited ones.

In a granular fixed bed, the incident radiation is partly absorbed, thus supplying the band-gap energy to the catalyst, and partly scattered by the catalysts particles themselves. Only a fraction of the scattered light meets again the catalyst and is either absorbed or scattered again. In the fixed catalyst film beds or in the in the catalyst coatings the scattered radiation will never again meet the catalyst after the first impact.

The probability for collisions with the scattered photons is higher if a mixing of catalyst particles is present. The fluidized bed catalytic reactors allow for an excellend contact between catalyst and the reagents, and a high mass and heath transfer velocity, besides an easy control of the reaction temperature. In photocatalytic reactions fluidized bed reactors can provide the advantage of a better use of the light radiation, with resulting increase of activity due to the absorption, by the photocatalyst, not only of the incident radiation, but also of the radiation scattered by the catalyst particles themselves.

The different overall reactor configurations can be compared by means of the illumination efficiency (Eq. 1), ηill, (Van Gerven T., Mul G., Moulijn J., Stankiewicz A., Chem. Eng. Process, 46 (2007) 781), which takes into account the catalyst illuminated surface per irradiated volume (k, m⁻¹), the average power efficiency (defined as the ratio of average incident radiant power on the catalyst, measured with a radiometric probe at different sites, to emitted radiant power), and the incident uniformity. The latter is often defined as the ratio of the catalyst surface that receives at least the minimum energy (i.e., the band-gap energy) and the total catalyst surface area

$${η}_{ill} = k ( \frac{{P}_{Cat}}{{P}_{Lamp}} ) ( \frac{{A}_{minE}}{{A}_{Cat}} ) \qquad Eq . 1$$

where ηᵢₗₗ is the illumination efficiency (m⁻¹), k is the catalyst illuminated surface per unit of irradiated reactor volume (m²ᵢₗₗ m⁻³_{reactor}, or m⁻¹), P_{Cat} is the radiant power incident on the catalyst surface (W), P_{Lamp} the radiant power emitted from the lamp (W), A_{min E} is the catalyst surface that receives at least the band-gap energy (m²) and A_{Cat} is the total catalyst surface (m²).

The catalyst illuminated surface per unit of irradiated reactor volume (k) takes values within the range 8500-170000 m⁻¹ in the case of slurry reactors. The most efficient reactors with regard to the illumination appear to be the microreactors, for which k reaches the value of 250000 m⁻¹. The totality of studies on photocatalytic reactions carried out in fluidized bed reactors employs conventional mercury UV lamps (with low and/or medium pressure) as the light source to activate the photocatalyst. In particular, for the photocatalytic treatment of nitrogen oxides (NOₓ) a fluidized bed of ultrafine particles of TiO₂ was applied (Matsuda S., Hatano H., Tsutsumi A., J. Chem. Eng. 82 (2001) 183). Three different TiO₂ particle agglomerates with primary particle diameters of 7, 20 and 200 nm, were used as the bed material.

The photocatalytic oxidation of NO on CuO-based catalysts loaded on titania support was carried out in annular two-dimensional fluidized bed reactors. With a CuO loading of 3.3 wt % the NO conversion in the modified two-dimensional fluidized bed photoreactor was more than 70% at 2.5 times the minimum fluidization velocity, U_{mf} (Lim T. H, Jeong S. M., Kim S. D., Gyenis J., J. Photochem. Photobiol. A: Chemistry, 134, (2000) 209).

The photocatalytic oxidation of ethanol vapour was investigated with an annular fluidized bed reactor (Kim M., Nam W., Han G. Y., J. Chem. Eng. 21 (2004), 721) employing silica gel powder coated with TiO₂. The UV lamp was installed at the center of the bed as the light source. It was found that at 1.2 times the U_{mf} (minimum fluidization velocity) value, about 80% of ethanol (with initial concentration of 10000 ppm) was decomposed, while an increase of superficial gas velocity reduced the reaction rate significantly.

Also photocatalytic NH₃ synthesis was successfully performed in a fluidized bed reactor on doped TiO₂. (Yue P. L., Khan F., Rizzuti L., Chem. Eng. Sci. 38 (1983), 1893).

In all cases, the photocatalyst should have good fluidization properties. The U.S. Pat. No. 5,374,405 to Firnberg et al: teaches a reactor comprising a rotating porous bed vessel drum within a plenum vessel. Gas is introduced through the walls of the drum and exits at the top. An ultraviolet light source is included within the drum. The U.S. Pat. No. 6,315,870 to Tabatabaie-Raissi et al. teaches a method for high flux photocatalytic pollution control based on the implementation of metal oxide aerogels in combination with a rotating fluidized bed reactor irradiated by an UV lamp placed along the rotation axis.

The U.S. Pat. No. 5,030,607 to Colmenares teaches a method for the photocatalytic synthesis of short chain hydrocarbons on UV light-transparent silica aerogels doped with photochemically active uranyl ions, in a fluidized bed photoreactor having one (1) transparent window and exposed to radiation from a light source external to the reactor.

The U.S. Pat. Appln. No. 2005/0178649 by Liedy relates to a system for carrying out photocatalysed reactions in liquid or gaseous reaction media, consisting of a reactor vessel with a solid particle photocatalyst, irradiated from the interior by mixing therein some microradiators. Said microradiators are excited by irradiation in a chamber external to the reaction vessel, and emit by fluorescence the radiation useful to the photocatalyst. The microradiators may then be separated from the photocatalyst and are recirculated to the external irradiation system.

With recent developments, there is great potential for UV-LEDs to become a viable light source for photocatalysis. A UV-LED is a diode, which emits UV-light by combining holes and electrons on the interface of two semiconductor materials. UV-LEDs are long-lasting, robust, small in size and high in efficiency. Their spectra are narrow and their peak wavelength can be located in selected positions by design.

The International Patent Application No. WO01/64318 by Kim et al. relates to a photocatalytic purifier adapted to eliminate various pollutants, such as volatile organic materials contained, in the air utilizing a photocatalyst. More particularly, the device employs a UV-LED to excite the photocatalyst, in the form of a fixed bed catalyst film coated in a carrier.

The International Patent Application No. WO2007/07634 by Muggli teaches a device for the indoor-air purification that utilizes a fluidized bed containing ultraviolet lights immersed in the catalyst bed to remove pollutants from indoor air. Fluidization aids, such as vibration and static mixers, may be employed to allow for better circulation of the catalyst bed to increase reaction rates.

No studies are known at present regarding the use of a two dimensional photocatalytic fluidized bed reactor, internally heated and irradiated by UV-LED arrays positioned at its external walls to realize photo-oxidation reactions. Further, no indications are known about the use of beds of catalyst diluted with alumina or silica or silica gel or glass of suitable particle size.

### OBJECT OF THE INVENTION

The main object of the invention is to develop a system for gas-solid heterogeneous photocatalytic reactions, which avoids the subsequent separation of the catalyst from the reaction stream.

The device consists of a two-dimensional fluidized bed reactor with two flat transparent walls with external irradiation, provided by UV lamps or UV-LED arrays. The fotoreactor is equipped with an electric heater immersed in the catalytic bed to control the reaction temperature up to 200° C.

Another object of the invention is to achieve the total photocatalytic oxidation of VOCs.

Another object of the invention is to demonstrate the effectiveness of the device in the selective photocatalytic oxidation of hydrocarbons.

A further object is to show the effectiveness of photocatalysts based on transition metals, anions such as sulphate, phosphate, etc., supported on aluminum or titanium or zirconium or zinc oxides or their mixed oxides, in specific photocatalytic reactions such as partial or total oxidation, and oxidative dehydrogenation.

### SUMMARY OF THE INVENTION

The present invention relates to the synthesis of organic compounds or the removal of volatile organic compounds (VOCs) by means of a fluidized bed gas-solid photocatalytic reactor with improved illumination efficiency. The proposed reactor consists of a two-dimensional fluidized bed catalytic reactor with two flat walls transparent to UV light, of a light system, preferably an UV-LED array, placed at the exterior of the two flat walls, and heated by Joule effect from the interior of the catalytic bed to control the reaction temperature. Through the choice of a suitable catalyse and of the working conditions of the fluidized bed photoreactor it is possible to carry out both total oxidation and partial oxidation reactions with high activity and selectivity. Surprisingly, the irradiated catalyst surface per unit of irradiated volume reaches values as high as 10⁶ m⁻¹, quite higher than the values proper of microreactors, which are about 250,000 m⁻¹, and of the slurry reactors, having values in the range of 8500-170000 m⁻¹. The photocatalytic system according to the present invention appears to have a high illumination efficiency due to the use of UV-LEDs, which allow for an irradiation in the direction orthogonal to the emission point, thus minimizing the loss of fotons.

The photoreactor efficiency was evaluated with regard to the oxidative dehydrogenation of cyclohexane to benzene and to cyclohexene, of ethylbenzene to styrene, of ethanol to acetaldehyde. It is to be noted that catalysts based on transition metals supported on TiO₂, Al₂O₃, ZrO₂, in the presence of sulfates or other anions, turned out to be more active in obtaining products of oxidative dehydrogenation or products of partial oxidation. The device is also effective in the total oxidation of benzene, acetone and toluene in diluted feeds.

More specifically, the present invention concerns the provision of a two-dimensional fluidized bed gas-solid reactor having two flat transparent walls with external illumination, supplied by two UV-LEDs arrays and characterized by a high illumination efficiency. The reactor is provided with an electric heater immersed in the catalyst bed to control the reaction temperature. The invention exploits the advantage of coupling the positive aspects dueto the use of a fluidized bed system with LEDs, which are robust, small in size and highly efficient in providing a light radiation of appropriate wavelength.

The fluidized bed reactor has 40 mm×10 mm cross-section, its height is 230 mm while its walls are 2 mm thick. A sintered metal filter (having a size comprised in the range 0.1-1000 μm, preferably in the range 4-50 μm and more preferably 5 μm size) is used for gas feeding to provide uniform gas distribution. Two arrays of LEDs were assembled and adapted to the fluidized bed photoreactor design in order to obtain the maximum reactor illumination efficiency. These LEDs have an emission spectrum centred at 365 nm, which is the right wavelength to activate the semiconductor employed as catalyst.

An objective of the invention is to realize the photocatalytic total oxidation of VOCs. Oxides of titanium, aluminum, zirconium, zinc, or their mixed oxide powders are used as catalysts. The addition of transition metals such as vanadium and molybdenum and/or anions such as sulphates or phosphates further enhances the desired properties of the photocatalyst. Transition metals and anions are supported by wet impregnation from aqueous solutions of salts suitably chosen, followed by treatment in air at high temperature.

Further, the present invention has been shown to be effective in the photocatalytic oxidation of hydrocarbons, in particular in the photocatalytic oxidative dehydrogenation. For the latter reaction, a wide variety of hydrocarbons such as cyclohexane, ethylbenzene and ethanol are fed to the fluidized bed reactor according to the invention. Supported molybdenum, vanadium and tungsten-based sulphated catalysts are preferably used. A variety of metal oxides such as titania, alumina and their mixed oxides are used as supports for active phases. Also in this case, transition metals and anions are supported by wet impregnation from different aqueous salt solutions suitably chosen, followed by treatment in air at high temperature.

### BRIEF DESCRIPTION OF THE DRAWINGS

FIG. 1 shows the schematic picture of the UV-LEDs array.

FIG. 2 shows a schematic picture of the two-dimensional photocatalytic fluidized bed reactor according to the invention.

FIG. 3 shows the scheme of laboratory apparatus for the measurement of photocatalytic activity.

FIG. 4 shows benzene conversion on TiO₂ (PC500), and on a catalyst containing 0.8 wt % V₂O₅ as nominal loading (0.8V) supported on PC500 as a function of irradiation time during photocatalytic oxidation in air. Experimental conditions: m_{catalyst}=3 g; C.°_{C6H6}=200 ppm; H₂O/C₆H₆ ratio=1.5; P=1 atm; T=80° C.; pH=3.9, Qₜₒₜ=50 Nlt/h; incident light intensity: 100 mW/cm².

FIG. 5 shows the evolution of carbon dioxide concentration formed during benzene photocatalytic oxidation in air stream on TiO₂ (PC500), and on a catalyst containing 0.8 wt % V₂O₅ as nominal loading (0.8V) supported on PC500 as a function of irradiation time. Experimental conditions: m_{catalyst}=3 g; C.°_{C6H6}=200 ppm; H₂O/C₆H₆ ratio=1.5; P=1 atm; T=80° C.; Qₜₒₜ=50 Nlt/h; incident light intensity: 100 mW/cm².

FIG. 6 shows the outlet reactor concentration (a. u.) of cyclohexane, oxygen, benzene and cyclohexene as a function of run time. Initial cyclohexane concentration: 1000 ppm; oxygen/cyclohexane ratio: 1.5; water/cyclohexane ratio: 1.6; Incident light: 100 mW/cm².

FIG. 7 shows the effect of incident light intensity and catalyst weight on steady state cyclohexane consumption rate obtained in the photocatalytic oxidative dehydrogenation of cyclohexane on 10 MoPC100 Al catalyst. Experimental conditions: C.°_{C6H12}=1000 ppm; O₂/C₆H₁₂ ratio=1.5; H₂O/C₆H₁₂ ratio=1.6; P=1 atm; T=120° C.; Qtot=50 Nlt/h.

FIG. 8 shows ethylbenzene conversion and styrene outlet concentration as a function of irradiation time on 8 Mo2 S catalyst. Experimental conditions: m_{catalyst}=14 g, C.°_{C8H10}=1000 ppm; O₂/C₈H₁₀ ratio=1.5; H₂O/C₈H₁₀ ratio=1.6; P=1 atm; T=120° C.; Qtot=50 Nlt/h; incident light intensity: 100 mW/cm².

FIG. 9 shows ethanol conversion and acetaldehyde outlet concentration as a function of irradiation time on a catalyst containing 5 wt % V₂O₅ as nominal loading supported on PC105. Experimental conditions: m_{catalyst}=2 g, C.°_{C8H10}=1 vol. %; O₂/ethanol ratio=2; P=1 atm; T=100° C.; Qtot=50 Nlt/h; incident light intensity: 100 mW/cm².

### DETAILED DESCRIPTION OF THE INVENTION

The main object of the invention is to develop gas-solid heterogeneous photocatalytic reactions for easy separation of the catalyst by the reaction stream. The device consists of a two-dimensional fluidized bed reactor with two flat transparent walls with external irradiation, provided by UV lamps or UV-LEDs arrays. The fotoreactor is equipped with an electric heater immersed in the catalytic bed to control the reaction temperature up to 200° C.

The two-dimensional fluidized bed reactor is designed in order to improve both the exposure of the catalyst to light irradiation and the mass and heat transport phenomena. Remarkably, through the choice of a suitable catalyst and fluidized bed photoreactor operating conditions it is possible to carry out both total and partial oxidation reactions with high selectivity. Even more remarkably, the illumination efficiency of the reactor is higher than that of other photoreactors previously reported.

The gaseous stream (with flow rate in the range 200-1000 Ncc/min, preferably in the range 500-830 Ncc/min and more preferably 830 Ncc/min) is introduced into the fluidized bed reactor through its rectangular cross section (40 mm×10 mm). The wall is made of transparent material and are 2 mm thick and 230 mm high. A porous filter of sintered metal (having a size in the range 0.1-1000 μm, preferably in the range 4-50 μm and more preferably 5 μm) is used for gas feeding to provide uniform gas distribution.

During transient condition, some catalyst elutriation phenomena can be observed.

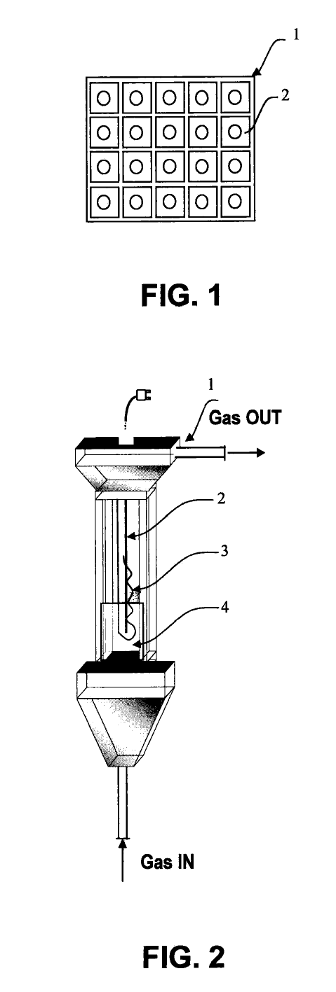

The reaction temperature is controlled by a PID controller connected to a heater system immersed within the catalytic bed. The reactor was illuminated by four UV mercury lamps with a power of 125 W each or by two UV-LEDs modules (Type NCCUO33 supplied by Nichia Corporation) positioned in front of the flat transparent windows. Each UV-LED array (FIG. 1) consisted of 20 units. The light intensity of the UV-LED operated at various forward currents is measured by an UV meter. The peak wavelength is 365 nm. A schematic picture of the fluidized bed reactor is shown in FIG. 2.

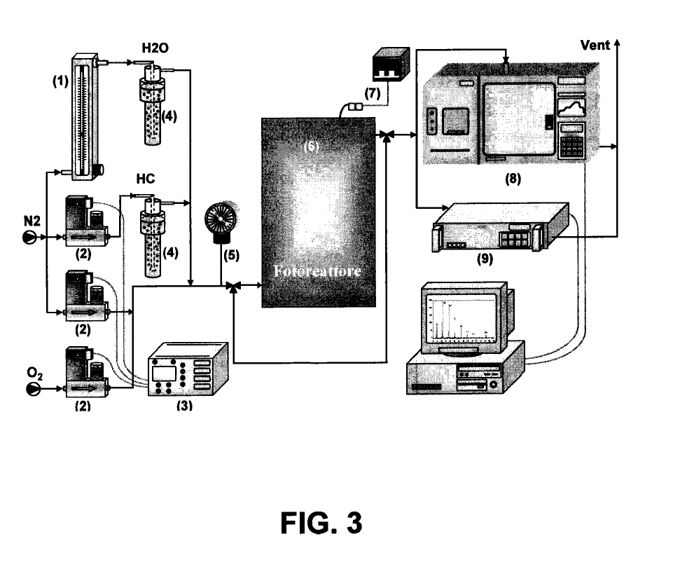

The gas flow rates were measured and controlled by mass flow controllers (supplied by Brooks Instrument). The gas composition was continuously measured by an on-line quadrupole mass detector (Trace MS, supplied by ThermoQuest) and by a CO—CO₂ NDIR analyser (Uras 10, supplied by Hartmann & Braun). The light sources are switched on after complete adsorption of the hydrocarbon on the catalyst surface. FIG. 3 reports a schematic picture of the laboratory apparatus for the measurement of photocatalytic activity.

An object of the invention is to realize the total photocatalytic oxidation of VOC. The device is effective in the total oxidation of a wide variety of organic pollutants such as acetone, toluene and benzene. Oxides of titanium, aluminum, zirconium, zinc, or their mixed oxide powders are used as catalysts. The addition of transition metals such as molybdenum, tungsten and vanadium and anions such as sulphates or phosphates further enhances the desired properties of the photocatalyst. Titania, alumina, zirconia, or mixed oxide powder can be used as supports.

The preparation procedure for catalyst samples containing various amounts of transition metals and of anions consists of two main steps. The first step is the impregnation of the support with an aqueous solution of the precursor salt of the oxyanion to support. The suspension is dried under stirring at 80° C. until complete removal of water. The oxyanion-doped sample is then obtained by calcination at 300° C. for 3 hours. The second step is the impregnation of the sample obtained from the 1^{st} step with an aqueous solution of precursor salt of the transition metal to be supported. Then the sample is dried at 120° C. for 12 hours and calcined at 400° C. for 3 hours. The oxyanion loading (expressed as SO₃ or P₂O₅ in the case of sulphate and phosphate respectively) is in the range 0.1-18 wt %, preferably in the range 0.2-5% and more preferably is 0.3 wt %. The transition metal loading (expressed as MoO₃, V₂O₅ or WO₃ in the case of molybdenum, vanadium or tungsten respectively) is in the range 0.2-10 wt %, preferably in the range 0.8-4% and more preferably is 0.8 wt %.

Photocatalytic activity tests were carried out feeding an air stream, with flow rate in the range 200-1000 Ncc/min, preferably in the range 500-830 Ncc/min and more preferably of 830 Ncc/min, containing steam and hydrocarbon at different concentrations (preferably in the range 100-1000 ppm, more preferably in the range 200-500 ppm, specifically 200 ppm. The water/hydrocarbon ratio is in the range 0-2 and more preferably 1.5. The reaction temperature is in the range 50-160° C., preferably in the range 70-120° C. and more preferably is 80° C.

The reactor is illuminated with an incident light intensity variable in the range 10-150 mW/cm², preferably in the range 30-120 mW/cm² and more preferably of 100 mW/cm². To improve the photocatalyst fluidization (and therefore the exposure to UV light), the latter is physically mixed with non-semiconductor solids belonging to the classes A and B of the Geldart distribution, preferably alumina and silica, more preferably α—Al₂O₃, silica gel or glass beads. The reactor is loaded with a mass of catalyst within the range 1-20 g, preferably in the range 2-4 g and more preferably with 3 g of catalyst. Surprisingly, photocatalytic activity tests show that the addition of transition metals and anions improves the properties of photocatalysts, making the system effective in the total oxidation of benzene, acetone and toluene in the presence of water vapour.

Another object of the present invention is to demonstrate the effectiveness of the device in selective photo-oxidation of hydrocarbons, in particular in the reaction of photo-oxidative dehydrogenation. For this latter reaction, a wide variety of hydrocarbons such as cyclohexane and ethylbenzene are fed to the fluidized bed reactor according to the invention. Catalysts based on transition metals (such as molybdenum, vanadium and tungsten) are preferably used. A variety of metal oxides such as titania, alumina, zirconia and their mixed oxides doped with anions (such as sulphate and phosphate) are used as supports for transition metals. The metal oxides are impregnated with a solution containing the precursor salt of the anion to support. The suspension is dried under stirring at 80° C. to complete removal of water excess. The doped sample is obtained by calcination at 300° C. for 3 hours. Thereafter, the doped sample is impregnated with an aqueous solution of precursor salt of the transition metal to be supported. Then the sample is dried at 120° C. for 12 hours and calcined at 400° C. for 3 hours.

Mixed oxides are obtained through the sol-gel method. For instance, TiO₂—Al₂O₃ mixed supports are prepared by dispersing the titania powder in a boehmite sol (obtained by acidifying a solid suspension of bohemite in bidistilled water). The system is then gelled by slight heating until it is too viscous to stir. The gel is thus dried at 120° C. for 3 hours and calcined at 500° C. for 2 hours. After calcination the solid is crushed and sieved to achieve a particle size suitable to fluidization (typically 50-90 μm). The mixed solid obtained is then impregnated with an aqueous solution of precursor salt of transition metal to be supported, dried and calcined at 400° C.

The oxyanion loading (expressed as SO₃ or P₂O₅ in the case of sulphate and phosphate respectively) is in the range 0.1-18 wt %, preferably in the range 0.2-6% and more preferably 2 wt %. The transition metal loading (expressed as MoO₃, V₂O₅ or WO₃ in the case of molybdenum, vanadium or tungsten respectively) is in the range 0.2-14 wt %, preferably in the range 2-12% and more preferably in the range 8-10 wt %.

Photocatalytic tests were carried out feeding nitrogen or helium stream (with flow rate in the range 200-1000 Ncc/min, preferably in the 500-830 Ncc/min and more preferably 830 Ncc/min) containing water and hydrocarbon at different concentrations (preferably in the range 100-50000 ppm, more preferably in the range 200-10000 ppm and specifically 1000 ppm) with an oxygen/hydrocarbon and water/hydrocarbon ratio in the range 0-10, preferably in the range 1-3 and more preferably 1.5 and 1.6 respectively. The reaction temperature was in the range 80-200° C., preferably in the range 90-140° C. and more preferably was 120° C.

The reactor was illuminated with an incident fotonic flux variable in the range 10-150 mW/cm², preferably in the range 30-120 mW/cm² and more preferably 100 mW/cm². The amount of catalyst loaded in the reactor was in the range 2-30 g, preferably in the range 3-25 g and more preferably in the range 14-20 g.

The proposed system has surprisingly proved effective in achieving the oxidative dehydrogenation of alkanes, cycloalkanes and alcohols, particularly the photo-oxidative dehydrogenation of cyclohexane to benzene and/or to cyclohexene and of ethylbenzene to styrene, as well as ethanol to acetaldehyde, with selectivity up to 100% to the desired products.

### EXAMPLES

Examples 1-4 show the results obtained for the measure of the photocatalytic activity on total oxidation and selective oxidation of hydrocarbons with evaluation of the illumination efficiency of the reactor in one exemplary case, employing both unsupported catalysts (TiO₂) and sulphated V₂O₅ and MoO₃-based catalysts supported on metal oxides (TiO₂ and γ—Al₂O₃ and their mixed oxides).

### Materials and Chemicals Used

Benzene with a purity grade equal to 99.9% was provided by Aldrich, toluene with a purity grade equal to 99.8% was provided by Aldrich, acetone with a purity grade equal to 99.8% was provided by Riedel de Haen, cyclohexane with a purity grade equal to 99.9% was provided by Aldrich and ethylbenzene with a purity grade equal to 99.9% was provided by Aldrich.

Ammonium heptamolybdate ((NH₄)₆ Mo₇O₂₄.4H₂O) was provided by J. T. Baker, ammonium metavanadate (NH₄VO₃) was provided by Carlo Erba Reagenti, ammonium sulphate ((NH₄)₂SO₄) was provided by Carlo Erba Reagenti.

TiO₂ (PC100 and PC500) samples were provided by Millenium Inorganic Chemicals. γ—Al₂O₃ (Puralox SBA 150) was provided by SASOL. Boehmite (Puralox SB1) was provided by SASOL.

### Example: 1

### Total Photocatalytic Oxidation of Benzene

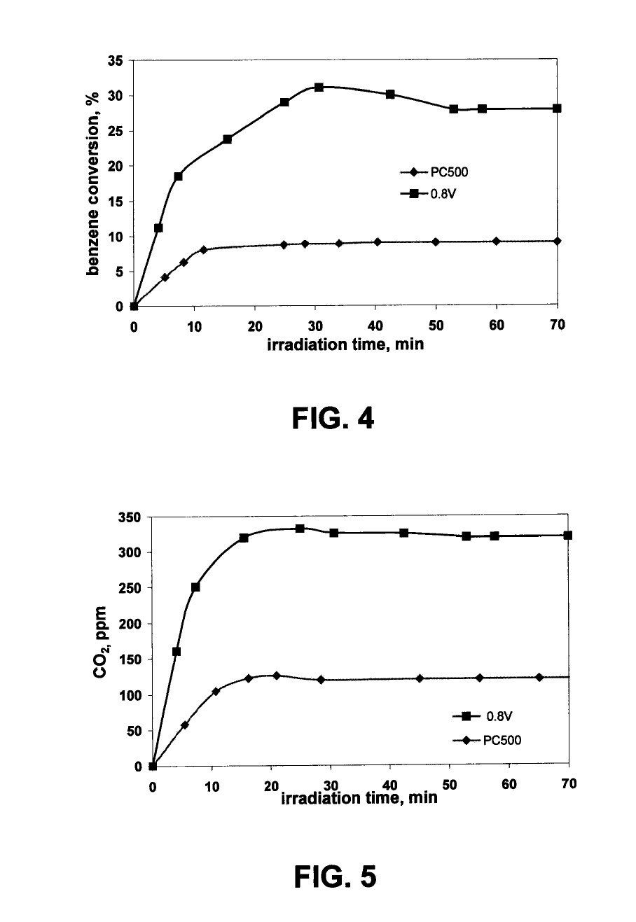

Photocatalytic oxidation of benzene was carried out feeding 830 (stp) cm³/min air containing 200 ppm of benzene in the presence of water vapour. Water/hydrocarbon ratio was equal to 1.5. The reaction temperature was 80° C. The reactor was loaded with 3 g of catalyst diluted with 6 g of α—Al₂O₃. The incident light intensity was 100 mW/cm². Benzene conversion and CO₂ outlet concentration on PC500, and on a catalyst containing 0.8 wt % of V₂O₅ nominal loading (0.8V) supported on PC500 as a function of irradiation time are reported in FIG. 4 and FIG. 5 respectively. CO₂ was the only product detected in the gas phase (100% selectivity), reaching steady state values after about 30 minutes. On 0.8V catalyst, steady state benzene conversion was about 28%, higher than that one obtained on PC500 (9%). No apparent deactivation has been observed under the experimental conditions. The addition of vanadium determined an increase of photocatalytic activity with respect unsupported titania.

### Example 2

### Oxidative Photocatalytic Dehydrogenation of Cyclohexane

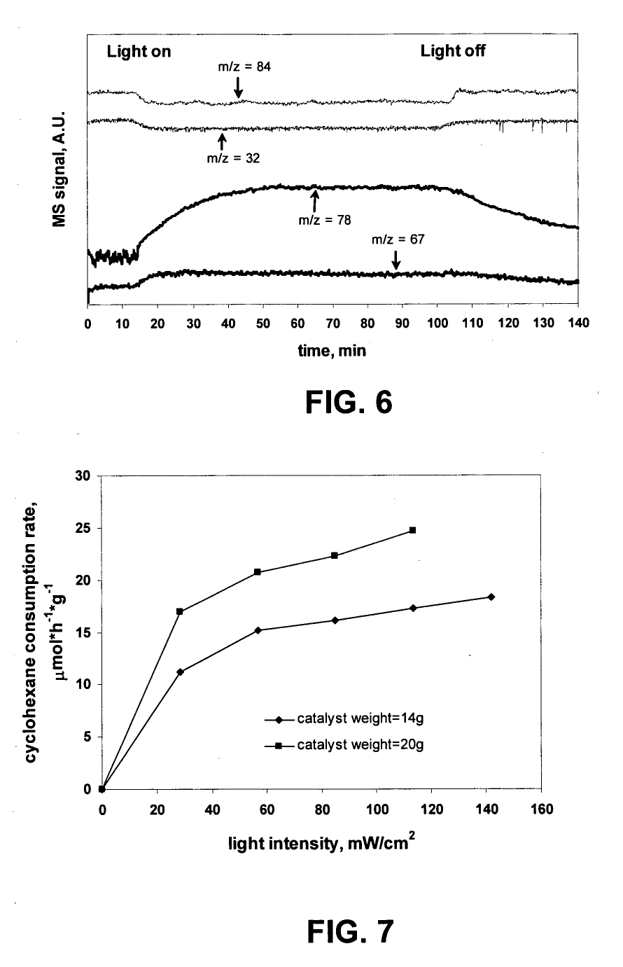

In FIG. 6 the results obtained by loading 14 g of a catalyst containing 10 wt % of MoO₃ nominal loading supported on TiO₂—Al₂O₃ (10 MoPC100 Al) are reported. TiO₂—Al₂O₃ mixed support was prepared by dispersing PC100 titania powder in a boehmite sol following the procedure reported in the detailed description of the invention. When the UV-LED modules were switched on, the cyclohexane outlet concentration immediately decreased reaching a steady state value corresponding to about 10% cyclohexane conversion after about 10 minutes. In the same figure the change of oxygen outlet concentration is also reported showing behaviour similar to that of cyclohexane.

The analysis of products in the outlet stream disclosed the presence of benzene and traces of cyclohexene, as identified from the characteristic fragments m/z=78, 77, 76, 74, 63, 52, 51, 50 (fragment 78 reported FIG. 6) and 82, 67, 54, respectively (fragment 67 reported in FIG. 6). No presence of carbon mono- and dioxide was disclosed, as detected by the NDIR analyser. The outlet concentration of benzene progressively increased reaching a steady state value after about 50 minutes. A similar trend was shown by cyclohexene concentration. No deactivation of catalyst was observed during photocatalytic tests.

To assess the effect of light intensity and of the catalyst weight on the photooxidative dehydrogenation of cyclohexane, the experiments were performed with a light intensity ranging between from (0 and 140 mW/cm²) and with two different weight of catalyst (14 and 20 g). The results are plotted in FIG. 7. Cyclohexane was unconverted in the absence of light and its reaction rate conversion increased up to about 25 μmol*h⁻¹*g⁻¹ in correspondence of a light intensity equal to 114 mW/cm² for a catalyst weight of 20 g. In all cases selectivity to benzene was higher than 99%.

The obtained results showed that it there no linear dependency between cyclohexane consumption rate and light intensity. Moreover the results reported in FIG. 7 evidenced the effect of catalyst weight on the photocatalytic activity. In particular, it increased by increasing the catalyst amount loaded into the reactor, as expected.

The value of k was estimated by measuring cyclohexane consumption rate on 10 MoPC100 Al sample as a function of catalyst weight. The irradiated volume was maintained unaltered by mixing the catalyst with the right amount of silica gel (which is transparent to UV light) giving the possibility to consider the ratio Pcat/Plamp equal to 1. Taking into account the obtained results with together the values of catalyst specific surface area (148 m²/g) and irradiated reactor volume (0.02 dm³), for photocatalytic reactor reported in this invention k is equal to 7.4*10⁶. Thus, by loading 14 g of catalyst into the reactor, the ratio A_{min E}/A_{cat} is equal to 0.043.

Finally, the value of ηill is 3.2*10⁶ which is higher than values reported for photocatalytic reactors (Van Gerven T., Mul G., Moulijn J., Stankiewicz A., Chem. Eng. Process, 46 (2007) 781).

### Example 3

### Photocatalytic Oxidative Dehydrogenation of Ethylbenzene to Styrene

The available literature does not report any scientific or patent publication concerning the use of a photocatalytic. An object of the invention is to demonstrate the effectiveness of the system in the selective photocatalytic oxidation of ethylbenzene to styrene which is one of the most important base chemicals in the petrochemical industry.

Photocatalytic activity tests were carried out on MoOₓ/γ—Al₂O₃ sample containing 8 wt ° A) of MoO₃ nominal loading and 2 wt % of SO₃ nominal loading. The photoreactor was fed with 830 Ncc/min N₂ stream containing 1000 ppm ethylbenzene, 1500 ppm O₂ and 1600 ppm H₂O. The reaction temperature and catalyst weight were 120° C. and 14 g, respectively. The incident light intensity was 100 mW/cm².

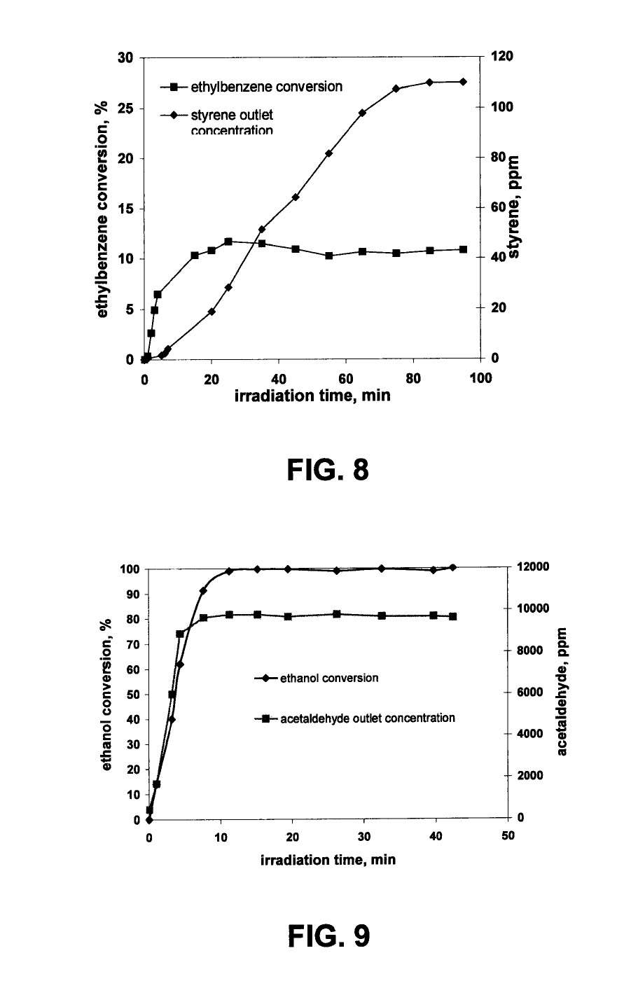

The only reaction product was styrene and no formation of CO₂ was detected. Ethylbenzene conversion and styrene outlet concentration are reported in FIG. 8. The steady state value of ethylbenzene conversion was reached after about 25 minutes and its value was about 11%. Styrene outlet concentration was 110 ppm after 85 minutes of illumination and increased less quickly with respect to ethylbenzene conversion. Total carbon mass balance was closed to 100% and no catalyst deactivation was observed.

### Example 4

### Photocatalytic Oxidative Dehydrogenation of Ethanol to Acetaldehyde

Recently, the oxidative dehydrogenation of ethanol to obtain high value added products is receiving increasing interest. The product for this type of reaction is acetaldehyde, which is industrially produced at a temperature of 500-650° C. (Ullmann, Encyclopedia of Industrial Chemistry, seventh edition (2004)).

According to the present invention the real possibility to achieve the selective oxidation of ethanol to acetaldehyde by means of a photocatalytic process is shown.

Photocatalytic oxidative dehydrogenation of ethanol was carried out feeding 830 (stp) cm³/min helium stream containing 1 vol. % of ethanol. Oxygen/ethanol ratio was equal to 2. The reaction temperature was 100° C. The reactor was loaded with 2 g of catalyst diluted with 4 g of silica gel. The incident light intensity was 100 mW/cm². Ethanol conversion and acetaldehyde outlet concentration on a catalyst containing 5 wt % of V₂O₅ nominal loading supported on PC105 as a function of irradiation time are reported in FIG. 9.

Ethanol conversion was total after about 12 minutes of irradiation. Correspondingly the concentration of acetaldehyde was equal to 9700 ppm with a selectivity of 97%. During the period of irradiation the formation of CO₂ and ethylene was found with selectivity of 2.8% and 0.2% respectively. Finally, no catalyst deactivation phenomena were observed.

### ADVANTAGES OF THE INVENTION

The foregoing shows that the invention disclosed involves the following advantages:
An easy and simple preparation of catalysts based on transition metals and sulphate anions supported on titanic and alumina for photo-oxidation reactions.
The activity of the catalysts for the photocatalytic removal of VOCs in gaseous stream.
The activity of the catalysts for the selective photo-synthesis of alkenes, aromatics and aldehydes in gaseous stream in mild conditions.
The ability to achieve high efficiency heterogeneous gas-solid photoreactions, avoiding the subsequent separation of the catalyst by the reaction stream.
The low volume of the two-dimensional photocatalytic fluidized bed reactor, with high illumination efficiency, and heated up to 160° C.
The extreme versatility in installation and use of one or more photoreactors in series or in parallel.
The high illumination efficiency also due to the use of UV-LEDs.

The present invention has been disclosed with particular reference to some preferred embodiments thereof but it is to be understood that modifications and changes may be brought to it without departing from its scope as recited in the appended claims.

1.-30. (canceled)

31. A two-dimensional photocatalytic fluidized bed reactor comprising a system with two flat transparent walls, a heating element positioned inside, irradiated from the outside by arrays of UV-LEDs, and a bed of catalyst as such or diluted with alumina and/or silica and/or silica gel and/or glass of suitable size.

32. A reactor according to claim 31, wherein the said catalyst is a transition metals and anions sulfate-based catalyst supported on titania or alumina.

33. A reactor according to claim 31, which is heated internally and irradiated from its transparent walls.

34. A reactor according to claim 31, wherein the said catalyst is diluted with alumina, diluted with silica gel or is in granular form.

35. A reactor according claim 31, which is irradiated from the out-side by two arrays of UV-LEDs.

36. A reactor according to claim 31, wherein the said catalyst is a sulfate and/or Mo, V based catalyst supported on titania or alumina.

37. A process for the photo-degradation of organic contaminants or for the selective partial oxidation of organic compounds comprising a treatment of the said organic contaminants or compounds in a two-dimensional photocatalytic fluidized bed reactor comprising a system with two flat transparent walls, a heating element positioned inside, irradiated from the outside by arrays of UV-LEDs, and a bed of catalyst as such or diluted with alumina and/or silica and/or silica gel and/or glass of suitable size, wherein the said treatment is carried out at ambient pressure and at a temperature between 40 and 160° C.

38. A process according to claim 37, for the total oxidation of organic compounds from the gas stream wherein the said catalyst is a sulfate and/or Mo, V based catalyst supported on titania or on cordierite.

39. A process according to claim 38, wherein said catalyst has a load of sulfate (expressed as SO₃) in the range 0.1-18%, more preferably in the range 0.2-5%, and has a load of Mo and/or V (as MoO₃ or V₂O₅) in the range 0.210%, more preferably in the 0.8-4%.

40. A process according to claim 37, for the photocatalytic oxidative dehydrogenation of organic compounds, wherein the said catalyst is a sulfate and/or Mo, V based catalyst supported on titania or on cordierite.

41. A process according to claim 40, wherein said catalyst has a load of sulfate (expressed as SO₃) in the range 0.1-18%, more preferably in the range 0.2-6%, and has a load of Mo and/or V (as MoO₃ or V₂O₅) in the range 0.2-14%, more preferably in the range 2-12%.

42. A process according to claim 37, for the photocatalytic oxidative dehydrogenation of organic compounds, wherein the said catalyst is a sulfate and/or Mo, V based catalyst supported on alumina or on cordierite.

43. A process according to claim 42, wherein said catalyst has a load of sulfate (expressed as SO₃) in the range 0.1-18%, more preferably in the range 0.2-6%, and has a load of Mo and/or V (as MoO₃ or V₂O₅) in the range 0.2-14%, more preferably in the range 2-12%.

44. A process according to claim 37, for the photocatalytic selective oxidation of organic compounds to aldehydes, wherein the said catalyst is a sulfate and/or Mo, V based catalyst supported on titania or on cordierite.

45. A process according to claim 44, wherein said catalyst has a load of sulfate (expressed as SO₃) in the range 0.1-18%, more preferably in the range 0.2-6%, and has a load of Mo and/or V (as MoO₃ or V₂O₅) in the range 2-10%, more preferably in the range 4-7%.

46. A process according to claim 37, wherein the said treatment is carried out in two or more of said two-dimensional photocatalytic fluidized bed reactors in series.

47. A process according to claim 37, wherein the said treatment is carried out in two or more of said two-dimensional photocatalytic fluidized bed reactors in parallel.
